(19) United States
(12) Patent Application Publication
(10) Pub. No.: US 2022/0301688 A1
(43) Pub. Date: Sep. 22, 2022

(54) GEOMETRIC PARADIGM FOR NONLINEAR MODELING AND CONTROL OF NEURAL DYNAMICS

(71) Applicant: UNIVERSITY OF SOUTHRN CALIFORNIA, Los Angeles, CA (US)

(72) Inventors: Maryam M. Shanechi, Los Angeles, CA (US); Han-Lin Hsieh, Los Angeles, CA (US)

(21) Appl. No.: 17/639,564

(22) PCT Filed: Oct. 8, 2020
(86) PCT No.: PCT/US2020/054848

§ 371 Date: Mar. 1, 2022

Related U.S. Application Data
(60) Provisional application No. 62/912,810, filed on Oct. 9, 2019.

(51) Int. Cl. G16H 30/20; G06F 30/27

(52) U.S. Cl. CPC G16H 30/20; G06F 30/27

(57) ABSTRACT

A method for nonlinear modeling, decoding, and control of neural dynamics includes identifying, based on neural time-series samples, a type of a manifold as a base for a neural model. The method further includes learning, based on a covering space, a dynamic model that is fit on the manifold to create the neural model. The method further includes creating, using the neural model, a geometric decoder and a geometric controller.

### CROSS-REFERENCE TO RELATED APPLICATIONS

This application claims the benefit and priority of U.S. Provisional Application No. 62/912,810, entitled “GEOMETRIC PARADIGM FOR NONLINEAR MODELING AND CONTROL OF NEURAL DYNAMICS,” filed on Oct. 9, 2019, the entire disclosure of which is hereby incorporated by reference in its entirety.

### STATEMENT AS TO FEDERALLY SPONSORED RESEARCH

This invention was made with government support under contract number N00014-19-1-2128 awarded by the Office of Naval Research. The government has certain rights in this invention.

### BACKGROUND

### 1. Field

The present disclosure relates to systems and methods for creating a geometric paradigm for nonlinear modeling, decoding, and control of neural dynamics, and for control of neural dynamics using the geometric paradigm.

### 2. Description of the Related Art

Emotional, cognitive, sensory, and motor functions and dysfunctions of humans and at least some primates arise from the nonlinear evolution of large-scale brain network activity over time, which may be referred to as “neural dynamics.” Precise control of these nonlinear brain network dynamics has not been achieved and their modeling and decoding remains challenging or impossible. Such modeling, if and when achieved, will have a profound impact across broad domains of basic neuroscience by advancing our understanding of neural mechanisms in health and disease, and causally dissecting the functional connections within brain networks and their role in driving behavior. Further, achieving control of these dynamics and the associated mental states will have immense impact on clinical neuroscience, and may revolutionize treatments for neuropsychiatric disorders such as depression, anxiety, addiction, and chronic pain. These disorders are a leading cause of disability of millions of patients who are not responsive to current treatments.

For example, in major depression, current treatments fail about 30 percent (30%) of patients, which amounts to at least 5 million patients in the United States (US) alone as of the year of filing of this disclosure (2020). To date, deep brain stimulation (DBS) for neuropsychiatric disorders has had variable efficacy, and clinical trials have not met their goals. DBS has so far been open-looped (i.e., it has applied a fixed pattern of stimulation without monitoring neural activity). Further, all prior DBS systems have lacked a model of neural dynamics and responses to stimulation.

Thus, there is a need in the art for systems and methods for nonlinear modeling, decoding, and control of neural dynamics.

### SUMMARY

Described herein is a method for nonlinear modeling, decoding, and control of neural dynamics. The method includes identifying, based on neural time-series samples, a type of a manifold as a base for a neural model. The method further includes learning, based on a covering space, a dynamic model that is fit on the manifold to create the neural model. The method further includes creating, using the neural model, a geometric decoder and a geometric controller.

Also described is system for nonlinear modeling, decoding, and control of neural dynamics. The system includes at least one of an input device or a sensor designed to receive neural time-series samples. The system further includes a processor coupled to the at least one of the input device or the sensor. The processor is designed to receive or determine a type of manifold to use as a base for a neural model. The processor is further designed to learn a dynamic model that is fit on the manifold to create the neural model based on a covering space. The processor is further designed to create a geometric decoder and a geometric controller using the neural model

### BRIEF DESCRIPTION OF THE DRAWINGS

Other systems, methods, features, and advantages of the present invention will be or will become apparent to one of ordinary skill in the art upon examination of the following figures and detailed description. It is intended that all such additional systems, methods, features, and advantages be included within this description, be within the scope of the present invention, and be protected by the accompanying claims. Component parts shown in the drawings are not necessarily to scale, and may be exaggerated to better illustrate the important features of the present invention. In the drawings, like reference numerals designate like parts throughout the different views, wherein:

FIG. 1 is a block diagram illustrating a system for generating a dynamic model of neural dynamics according to an embodiment of the present invention;

FIG. 2 is a block diagram illustrating a system for stimulating a brain based on the dynamic model of FIG. 1 according to an embodiment of the present invention;

FIG. 3 is a flowchart illustrating a method for nonlinear modeling, decoding, and control of neural dynamics according to an embodiment of the present invention;

FIG. 4 is a drawing illustrating the major steps of a geometric paradigm for neural dynamics according to an embodiment of the present invention;

FIG. 5 illustrates a transformation from a bounded plane to a torus according to an embodiment of the present invention;

FIG. 6 illustrates how a trajectory on a distorted nonlinear manifold can be obtained from an equivalent trajectory on a covering space according to an embodiment of the present invention;

FIG. 7 illustrates an example geometric dynamic model according to an embodiment of the present invention;

FIG. 8 illustrates a partition of a function to map a manifold state to neural samples on a distorted torus according to an embodiment of the present invention;

FIG. 9 illustrates a geometric controller according to an embodiment of the present invention;

FIG. 10 illustrates closed-loop control of neural activity using a geometric controller according to an embodiment of the present invention;

FIG. 11 illustrates two three-dimensional trajectories over two different manifold types but with the same two-dimensional projection according to an embodiment of the present invention;

FIG. 12 illustrates an exemplary implementation of the method of FIG. 3 according to an embodiment of the present invention;

FIG. 13 illustrates results of an exemplary use of the method of FIG. 3 according to an embodiment of the present invention;

FIG. 14 illustrates that decoding mood is possible in principal and projects that the geometric decoder can better decode mood than conventional methods according to an embodiment of the present invention; and

FIG. 15 illustrates that predicting the neural response to stimulation is possible in principal and projects that the geometric input-driven dynamic model can better predict the response than conventional linear methods according to an embodiment of the present invention.

### DETAILED DESCRIPTION

A standing challenge in neuroscience is to control the activity of large populations of interconnected neurons that underlie our brain's functions and dysfunctions. The dynamics of these high-dimensional neural activity patterns—i.e., how they evolve in time—are immensely complex and nonlinear, making their modeling extremely difficult. Thus, to date, the precise control of neural dynamics and the associated mental states using stimulation input has not been achieved. If it was possible to accurately model nonlinear neural dynamics and control them, this would both elucidate the neural basis of behavior and also be usable to treat prevalent and disabling mental disorders such as depression, anxiety, addiction, or the like, which each contribute to disability worldwide. The present disclosure provides systems and methods for accurately modeling, decoding, and controlling nonlinear neural dynamics.

The present disclosure describes a novel biologically-informed geometric paradigm to achieve nonlinear dynamic modeling and closed-loop control of neural population activity. The present disclosure further provides results of experiments using the geometric paradigm on brain network activity collected from a monkey motor system to characterize the neural dynamics of complex movements. The same geometric paradigm has application to control the neural biomarkers of depressed mood based on human corticolimbic activity. This geometric paradigm will be based on a central idea: if it is possible to learn and model the low-dimensional nonlinear geometric space (i.e., nonlinear manifold) over which neural population activity evolves in time, it is also possible to analytically write the dynamic model in a much simpler form over this manifold. This will allow for precise modeling of nonlinear dynamics and enable their control, which is impractical without writing the dynamic model in simpler form.

To date, the geometry of the space over which neural population dynamics evolve has not been considered in dynamic models. This central idea presents a major innovative departure from all current neural dynamic modeling paradigms, which either fit complex recurrent neural networks that are hard to interpret and not amenable to control or which describe the dynamics linearly over hyperplanes. Indeed, there is evidence that rotational neural dynamics exist in motor cortices, olfactory cortices, and visual cortices. It is hypothesized that these rotations are observed because the inherent space over which neural population activity evolves is a nonlinear manifold with loops and holes (such as a loop or torus) rather than a linear hyperplane. The present disclosure develops a new geometric paradigm necessary to achieve precise control of nonlinear neural dynamics for the first time by identifying the manifold and incorporating biologically-informed nonlinearities. This is achieved by combining modern tools from each of algebraic topology, differential geometry, stochastic control, and neurophysiology.

This paradigm has two broad areas of impact. The first is related to translational neurotechnology: the geometric controllers described herein will help revolutionize treatments for neuropsychiatric disorders such as depression or addiction or chronic pain or the like for millions of patients by achieving model-based closed-loop control of mental states with electrical brain stimulation, which is unrealized to date. The second broad area of impact is neurophysiology: the approach described herein will provide a new tool to build nonlinear models of neural dynamics, which are interpretable and thus can elucidate the neural basis of behavior and disease. Also, the geometric controllers provide a novel tool to uncover functional causality in brain networks.

The novel geometric paradigm disclosed herein is usable to: (1) identify the type of manifold that embeds neural dynamics (e.g., a torus or a sphere) using topological data analysis (TDA); (2) develop novel algorithms that learn functions to regress neural activity to the low-dimensional nonlinear manifold, and fit analytical dynamic models over this manifold (both with and without stimulation input); and (3) build decoders and controllers of neural dynamics that incorporate the biologically-informed nonlinear geometric models. Application of the paradigm with both Monte Carlo simulated data as well as rich data from the brain demonstrated the effectiveness of the paradigm. The dataset was previously-recorded spike-field network activity from the motor cortices of non-human primates (NHP) performing a 3D complex reach-and-grasp task. The paradigm can also be applied on multisite intracranial electrocorticogram (ECoG) network activity in the human corticolimbic system with simultaneous tracking of mood and with electrical stimulation.

Emotional, cognitive, sensory, and motor functions and dysfunctions of humans and at least some primates arise from the nonlinear evolution of large-scale brain network activity over time, which may be referred to as “neural dynamics.” Prior to this disclosure, precise control of these nonlinear brain network dynamics has not been achieved and their modeling remains challenging or impossible. Achieving accurate modeling and control of neural dynamics and the associated functions will have a profound impact across broad domains of basic neuroscience by advancing our understanding of neural mechanisms in health and disease, and causally dissecting the functional connections within brain networks and their role in driving behavior. Further, achieving control of nonlinear brain network dynamics and the associated mental states will have immense impact on clinical neuroscience and can revolutionize treatments for neuropsychiatric disorders such as depression, anxiety, addiction, and chronic pain, which are a leading cause of disability of millions of patients who are not responsive to current treatments.

For example, in major depression, which is the most disabling of these disorders, current treatments fail about 30 percent (30%) of patients, which amounts to at least 5 million patients in the United States (US) alone as of the year of filing of this disclosure (2020). As symptoms of emotional dysregulation are manifestations of abnormal activity in large-scale brain networks, selective control of this abnormal activity with electrical stimulation may provide a transformative treatment. To date, deep brain stimulation (DBS) for neuropsychiatric disorders has had variable efficacy, and clinical trials have not met their goals. DBS has so far been open-looped (i.e., it has applied a fixed pattern of stimulation without monitoring neural activity). Further, all prior DBS systems have lacked a model of neural dynamics and responses to stimulation. The innovative idea disclosed herein provides guiding stimulation not only by feedback of neural activity to close the loop, but also by providing a geometric model of the nonlinear neural response to enable control of mental states with unprecedented precision.

Even in terms of dynamic modeling alone, the geometric paradigm of the present disclosure introduces a major innovative departure compared to all current modeling paradigms by identifying the nonlinear geometry and learning the dynamic model over the nonlinear geometry. To date, neural dynamic models have been largely linear, meaning that they have used linear dimensionality reduction methods such as principal component analysis (PCA) on neural population activity and have learned linear state descriptions over hyperplanes. While useful for studying simple or constrained tasks in experiments, linear models cannot capture neural activity patterns underlying unconstrained naturalistic behavior that are likely much more complex and nonlinear. Thus, modeling nonlinear dynamics are critical for two goals: (i) elucidating the true underlying neural mechanisms of behavior and disease, and (ii) enabling their control. However, in order to achieve these goals, a nonlinear model should not only explain large variance in neural dynamics but also be (1) interpretable, (2) low-dimensional, and (3) controllable. Increasing the explained variance while satisfying these three properties are competing objectives that make the modeling extremely challenging. For example, recurrent neural networks (RNNs) can improve neural decoding but such RNNs are hard to interpret due to having thousands of parameters, due to having a high state dimension (e.g., hundreds of dimensions), and due to no controller existing for them given their complex form. The nonlinear geometric paradigm of the present disclosure resolves the challenge for the first time: the geometric paradigm explains the large neural variance and is simultaneously interpretable, low-dimensional, and controllable. A major innovation that facilitated meeting these competing objectives was discovery of a low-dimensional interpretable geometry first, and then discovery of a dynamic model in much simpler form over the geometry.

In terms of control, precise control of brain network dynamics has not been achieved to date given their high dimensionality and nonlinearity. This is because even for a known nonlinear model, deriving a controller is hard or intractable. The problem gets worse for neural data because they have unknown nonlinearity whose modeling, as explained above, is challenging. Also, for control, deriving nonlinear dynamic models of the neural response to stimulation is critical, but is yet elusive. The geometric approach of the present disclosure presents an innovative paradigm shift in dynamic brain network modeling to study neural mechanisms and, for the first time, enables model-based closed-loop control of nonlinear brain dynamics.

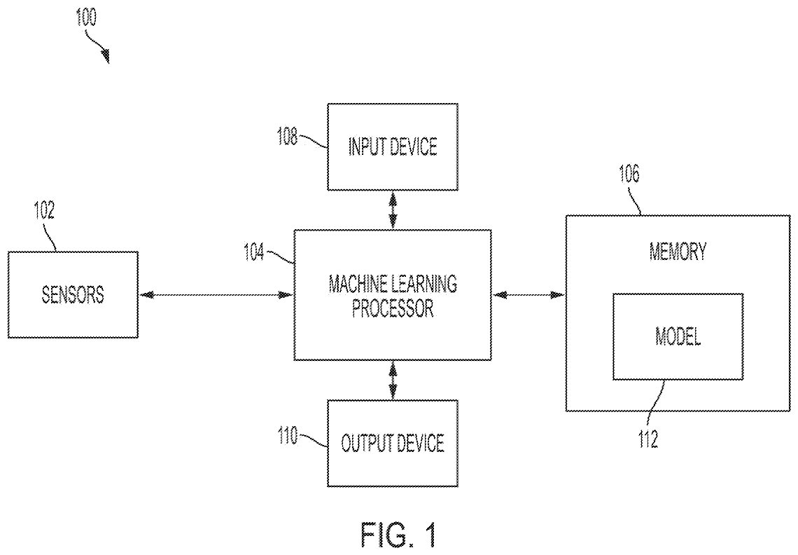

Referring now to FIG. 1, a system 100 for creating a dynamic model 112 of neural response to stimulation is shown. The system 100 may include one or more sensor 102, a machine learning processor 104, a non-transitory memory 106, an input device 108, and an output device 110. The one or more sensor 102 may include any sensors such as sensors capable of detecting electrical brain wave data. In that regard, the sensor 102 may be directly or indirectly coupled to a brain and may detect data corresponding to electrical brain activity of the brain or any other measurement modality from the brain.

The machine learning processor 104 may include any processor or controller capable of performing machine learning functions. For example, the machine learning processor 104 may include an application specific integrated circuit (ASIC), a digital signal processor (DSP), a general purpose processor, a field programmable gate array (FPGA), or the like. The machine learning processor 104 may perform logic functions based on instructions, such as instructions stored in the memory 106.

The memory 106 may include any non-transitory memory capable of storing data usable by the machine learning processor 104. For example, the memory 106 may store instructions usable by the processor to perform functions. As another example, the memory 106 may store data as instructed by the machine learning processor 104.

The input device 108 may include any input device such as a mouse, keyboard, button, a touchscreen, or the like. In some embodiments, the input device 108 may include a data port capable of receiving data from a separate device (e.g., a universal serial bus (USB) port, a Wi-Fi port, a Bluetooth port, an Ethernet port, or the like).

The output device 110 may include any output device such as a speaker, a display, a touchscreen, or the like. In some embodiments, the output device 110 may include a data port capable of transmitting data to a separate device.

The machine learning processor 104 may receive signals from the sensors 102 (or previously-recorded signals from the input device 108). The machine learning processor 104 may further receive additional information such as mood states associated with the signals, motor activities (e.g., raising a hand, smiling) associated with the signals, or the like. The machine learning processor 104 may further receive additional information such as stimulation signals including electrical or optogenetic stimulation applied to the brain, visual or auditory stimuli presented to the subject, or the like. Based on the received signals and data received by the input device (and as discussed in greater detail below), the machine learning processor 104 may create a dynamic model 112 of neural response to stimulation and how neural activity represents a brain state, function or dysfunction such as a mood or movement.

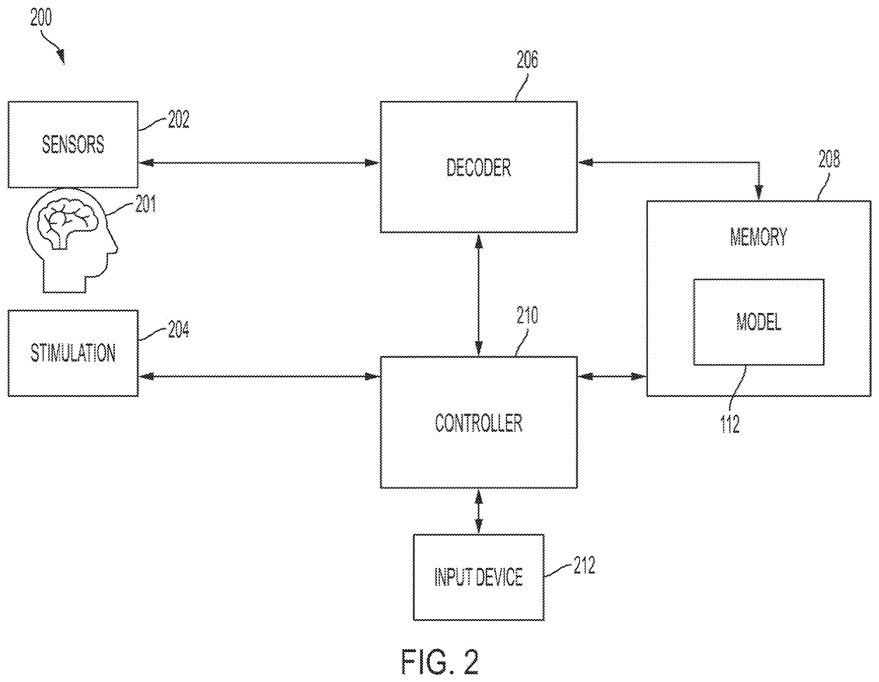

Referring now to FIG. 2, a system 200 for applying the dynamic model 112 to a brain 201 is shown. The system 200 includes one or more sensor 202, one or more stimulation device 204, a decoder 206, a memory 208, a controller 210, and an input device 212. The one or more sensor 202 may be the same or different sensor as the sensor 102 of FIG. 1. The stimulation device 204 may be integrated with the sensor 202 or may be separate device. For example, the stimulation device 204 may include probes or electrodes designed to transmit electrical signals to specific portions of the brain 201 or to deliver light for optogenetic stimulation.

The decoder 206 includes a geometric decoder capable of decoding the recorded neural activity from the brain 201 using the dynamic model 112. In that regard, the geometric decoder may include a logic device. The decoder will decode a desired brain state such as a mood or movement, or the like.

The memory 208 may include any non-transitory memory, and may store the dynamic model 112 therein.

The controller 210 may include any controller capable of generating signals to be applied to the brain 201 by the stimulation device 204 based on the decoded brain state, the model 112, and a target brain state.

The input device 212 may include any input device capable of receiving input such as a keyboard, mouse, touchscreen, or the like. The input device 212 may receive a target brain state that is desired of the brain 201.

In operation, the sensors 202 may detect neural activity from the brain 201. The neural activity detected by the sensors 202 may include any form of neural activity such as electrical signals, optical signals, or the like. The decoder 206 may decode the brain state of the brain 201 based on the detected neural activity. The controller 210 may receive the decoded brain state from the decoder 206, the target brain state from the input device 212, and the dynamic model 112 and may stimulate the brain 201 via the stimulation device 204 in order to achieve the target brain state.

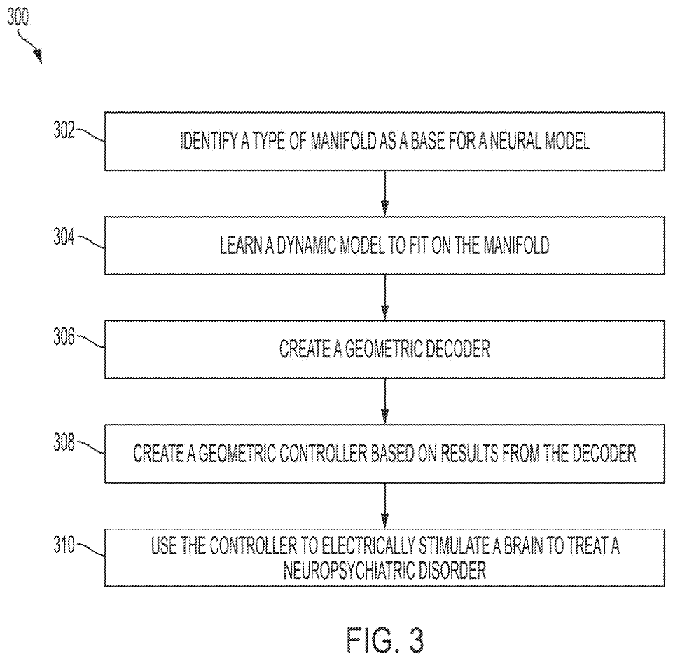

Referring now to FIG. 3, a flowchart illustrates a high-level method 300 for nonlinear modeling, decoding, and control of neural dynamics. Additional details regarding the method 300 will be described more fully below with reference to later FIGS. The method 300 may be implemented using a system such as the system 100 of FIG. 1, the system 200 of FIG. 2, or a combination thereof.

The method 300 may begin in block 302 where a type of manifold to be used as a base for a neural model is identified. In block 304, a dynamic model is learned to fit on the identified manifold of block 302. In block 306, a geometric decoder is created. The geometric decoder may be usable to decode brain states based on neural activity. In block 308, a geometric controller is created based on results from the decoder of block 306. In block 310, the controller is used to electrically stimulate a brain to treat a neuropsychiatric disorder, or for another goal.

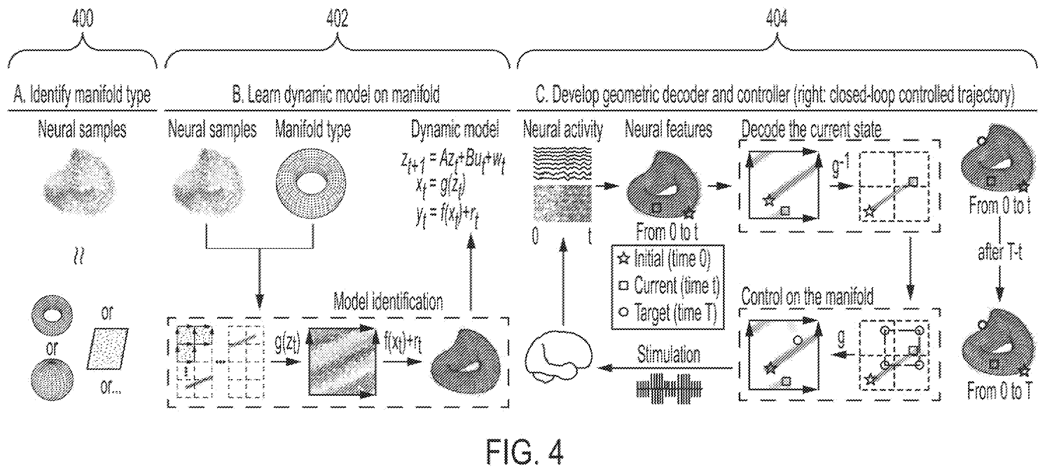

Referring now to FIG. 4, a high-level diagram illustrates exemplary implementation of the method 300 of FIG. 3. In particular, the geometric paradigm includes three main steps. The first step 400 is to identify the type of manifold from recorded data using topological data analysis. The second step 402 is to learn the dynamic model on the nonlinear manifold. Any trajectory on the manifold has an equivalent trajectory over a hyperplane that is called a covering space. The third step 404 is to develop a geometric decoder and a geometric closed-loop controller on the covering space. The closed-loop controller will move the neural features on the low-dimensional nonlinear manifold towards their target values, as shown on the right of the third step 404.

The ideas provided herein includes first identifying the type of low-dimensional nonlinear manifold over which brain network activity evolves, and then developing new methods to account for this nonlinear geometry in modeling and control. Why is it important to model the geometry? First, this will find nonlinearities that are informed by biology, are low-dimensional, and are interpretable rather than generic and high-dimensional. Second, this will build nonlinear models of neural dynamics that are accurate yet of sufficiently low complexity so as to enable model-based control of mental states. Third, if the nonlinearity is not modeled in the geometry, it should be built entirely in the dynamic model which is difficult and, even if possible, makes control intractable. The present disclosure first outlines a way to write the dynamic model analytically over a manifold type identified with TDA. The disclosure then outlines novel methods to learn the dynamic model over the identified manifold—a main unresolved challenge in data science.

How does knowing the manifold type help build the dynamic model for neural activity? Most known nonlinear manifolds found in nature, while complex, can be equivalently described on a hyperplane that is referred to as a “covering space.” An idea of the present disclosure is that if a transform between the nonlinear manifold and its associated covering space is learned, a complex neural trajectory on the nonlinear manifold can be mapped to an equivalent much simpler one over a hyperplane. As the nonlinearity is now captured through the manifold geometry, simple linear dynamic models for the equivalent trajectory can be fit over the covering space for the manifold, and the decoder and controller can be designed over this hyperplane, which becomes tractable.

The approach may be visualized using a simple but representative example. Suppose 3 neural signals are recorded (e.g., firing rate of 3 neurons or 3 local field potential signals or 3 ECoG signals). Coordinated network dynamics often have much smaller dimension than the number of recorded signals. Assuming that the activity is 2-dimensional (2D) and evolves on a 2D nonlinear manifold, the manifold could belong to a diverse class (e.g., sphere, Klein bottle, torus) with any distortion (think of a torus made of clay and distorted in any of a number of manners). Here, it is assumed that the manifold is a distorted torus.

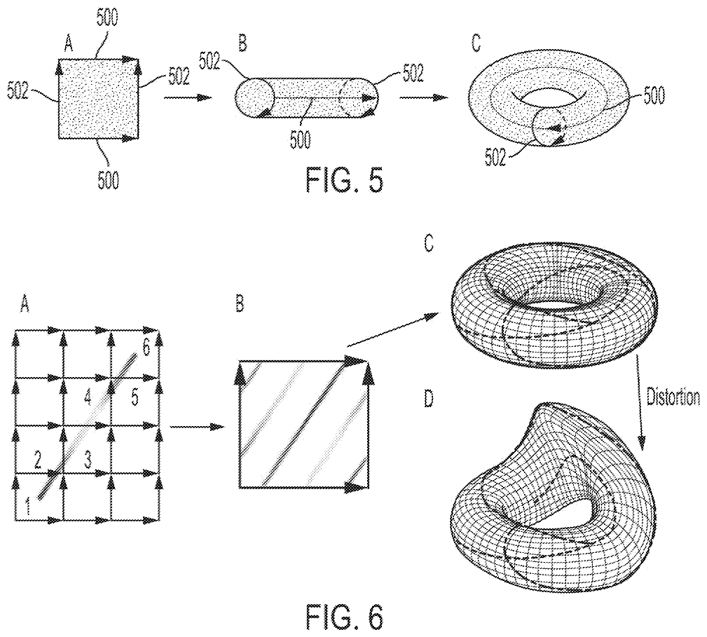

How can a complex nonlinear trajectory of brain network activity be described on a plane? FIG. 5 shows how a torus (C) is equivalent to a bounded plane (A). If the opposite boundaries of the bounded plane in the direction of the arrows are connected (i.e., first connect boundaries 500 as in B and then connect boundaries 502) the result is the torus shown in C. This way, any trajectory over the torus (C) can be represented equivalently on the bounded plane (A).

FIG. 6 illustrates how a trajectory on a distorted nonlinear manifold can be obtained from an equivalent trajectory on the covering space. Equivalent trajectories are shown on the covering space (A), bounded plane (B), torus (C), and distorted torus (D). Evolution in time is shown as a gradient. A to B: Map covering space to bounded plane by putting the 6 blocks containing the trajectory on top of each other. B to C: map covering space to torus as in FIG. 5. C to D: Apply a distortion. A line on the covering space is a spiral on the torus, with any distortion. Thus complex trajectories can be described in much simpler form on the covering space.

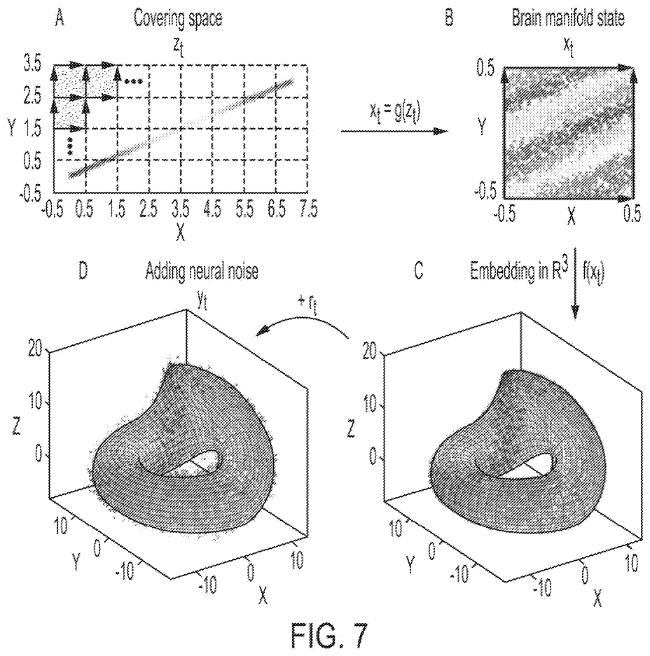

While easier than a torus, drawing a trajectory on a bounded plane is still complex as it is necessary to go to the opposite side when a boundary is reached (FIG. 6, B). This complexity is resolved using the idea of covering space (FIG. 6, A). To build a covering space, the plane ( [symbol] ²) will be filled with many bounded planes and a trajectory will be drawn over it. If all component bounded planes that contain part of the trajectory (blocks 1 to 6 in FIG. 6, A) are placed on top of each other, the equivalent trajectory is obtained on the original bounded plane (FIG. 6, B). The equivalent trajectory on a torus (FIG. 6, C) can now be obtained by connecting the boundaries of the bounded plane (FIG. 5). Any distortion can also be applied to get more flexible trajectories (FIG. 6, D). Thus, an analytic map between the plane and torus is obtained. This process allows for analysis of any complex trajectory on any distorted nonlinear manifold from a hyperplane (i.e. the covering space), which is much more tractable for modeling, decoding, and control. Now the idea of the covering space is applied to write an analytic model for nonlinear temporal dynamics of a network of n neural channels whose activity is embedded in a d-dimensional (d-D) manifold as provided in Equations 1-3 below and shown in FIG. 7. FIG. 7 illustrates an exemplary geometric dynamic model. From A to D, the state zₜ is mapped to noisy neural signals yₜ. Each dot is the observed triplet of neural signals at one time point. Dot density evolution represents time evolution.

zₜ₊₁=Azₜ+Buₜ+wₜ Equation 1:

xₜ=g(zₜ) Equation 2:

yₜ=ƒ(xₜ)+rₜ Equation 3:

In Equations 1-3, yₜ is the n-D activity at time t (FIG. 7, D; firing rates, ECoG features). xₜ is the d-D brain state on the manifold (FIG. 7, B). Map ƒ describes how the manifold is embedded in [symbol] ⁿ (FIG. 7, C). zₜ is the equivalent d-D state on the covering space with a covering map g (FIG. 7, A), which describes the operation of putting all bounded planes on each other. uₜ is input (e.g., electrical stimulation). A and B are parameters. rₜ and wₜ are white Gaussian noises with covariances R and W.

In FIG. 7, d=2 and n=3. For visualization only, state zₜ follows a line with some noise wₜ (FIG. 7, A). Even this simple dynamic model on the covering space can describe very complex dynamics for the brain network activity (FIG. 7, D) as the nonlinear distorted geometric space of neural activity is modeled. Note yₜ is not on the distorted torus, but close to it (FIG. 7, D). Machine learning methods in the paradigm described below learn the model elements ƒ, g, A, B, R, W from neural data yₜ, and then the model is used to decode the brain state xₜ (e.g., motor or mood state) from yₜ, and to control this brain state by choosing uₜ in closed-loop based on the model and neural feedback. Now each step of this innovative paradigm will be outlined in detail.

The first part of the discussion regards identification of the type of manifold from neural data. The type of nonlinear manifold is identified from neural time-series samples y_{1:T}={y₁, . . . , y_{T}} This manifold type identification is a major departure from current methods on dynamic modeling, which assume that the brain state evolves linearly on linear hyperplanes whose embedding function ƒ⁻¹ is calculated by principal component analysis (PCA), and do not model the geometry. Many common manifolds can be described using a covering space (e.g., torus, Klein bottle, 3-sphere) and can be used to write the dynamic model in Equations 1-3. Finding the manifold type from data is difficult in general. Here, based on the biological evidence that neural dynamics (motor, olfactory, visual) have rotations, the present disclosure hypothesizes that the neural space is a nonlinear manifold with loops/holes. Thus, the manifold type can be found by counting the number of persistent holes or loops in data, using a method termed TDA. TDA counts this number by computing Betti numbers bᵢ (i=1:∞), which are topological properties and don't change under different distortions or ƒ functions (distorting a torus does not change its Betti numbers; it is still fundamentally a torus). For example, for a torus, b₀=1, b₁=2, b₂=1 and 0 elsewhere, as it is a connected object (b₀=1), with two 1D loops (the circles in FIG. 5, C; b₁=2) and a tube-like hole covered by the torus surface (b₂=1).

The next part of the discussion is regarding learning of the geometric dynamic model on the identified manifold. Although identifying the manifold type from TDA in many brain areas such as the motor or corticolimbic systems studied here will be a breakthrough in itself, to enable dynamic modeling and control it may be necessary to go beyond TDA. Even if this manifold is known, learning a dynamic model on it—that analytically writes future neural activity as a function of its past—and building a controller on it may present unresolved challenges. While TDA has been used for qualitative description or for classification/decoding (cardiac arrhythmias, head direction), to date, TDA has not been linked to quantitative dynamic modeling or to control in data science. The present disclosure develops new methods to enable this link for the first time in step 2 using the idea of covering space. It may be necessary to learn all unknown functions/parameters in Equations 1-3 from neural samples y_{1:T}. First, the covering map g and function ƒ are learned (below); then parameters A, B, W, R are found by deriving new unsupervised expectation-maximization (EM) methods, for example. The covering map g is given by the manifold type from TDA (step 1) as most common manifolds have a known g.

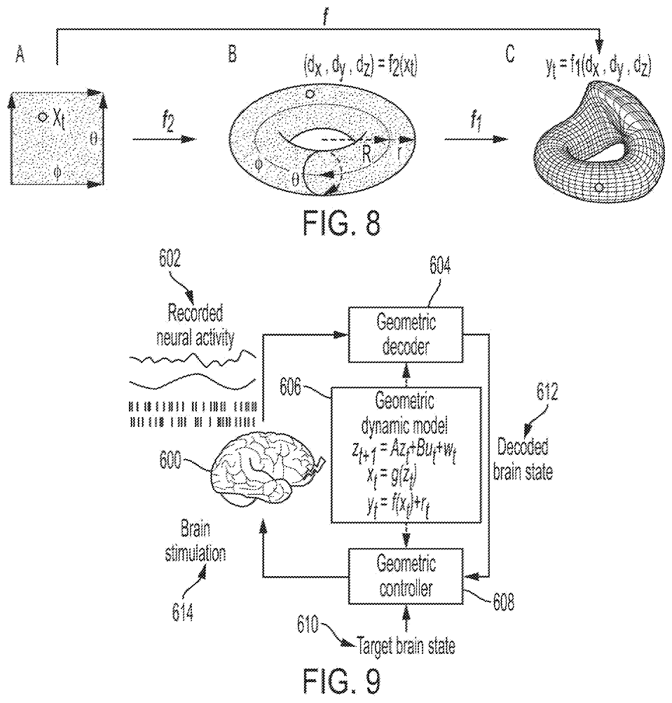

Thus a difficult step is to learn ƒ, for which the present disclosure devised two alternative methods. First is to decompose ƒ into two maps: ƒ₁ from undistorted coordinates (dₓ, d_{y}, d_{z}) to neural data yₜ (FIGS. 8, B and C) and ƒ₂ from the manifold state xₜ to the embedding space (FIGS. 8, A and B). In particular, FIG. 8 illustrates a partition of ƒ=ƒ₁∘ƒ₂ to map the manifold state to the neural samples on the distorted torus. ƒ₂ can be written based on the manifold type found by TDA, e.g., ƒ₂ for torus coordinate (dₓ, d_{y}, d_{z}) in [symbol] ³ (FIG. 8, B) based on bounded plane coordinates θ, ϕ∈[0,2π] (FIG. 8, A) are given by Equations 4-6 below.

dₓ=(R+r×cos θ)×cos ϕ Equation 4:

d_{y}=(R+r×cos θ)×sin ϕ Equation 5:

d_{z}=r×sin θ Equation 6:

In Equations 4-6, r, R are the two radii (FIG. 8, B). With this decomposition, the next step is to learn ƒ₁, the distortion function. When the manifold is a d-D hyperplane [symbol] ^{d}, ƒ₁ can be learned by PCA. But it may still be necessary to regress neural samples to a nonlinear manifold. To learn ƒ₁, existing nonlinear dimensionality reduction (NDR) may be used such as Isomap and local linear embedding (LLE) and may be combined with support vector methods of functional approximation of various kernels. To date, NDR methods have largely learned nonlinear manifolds without loops/holes, i.e., those isomorphic to a hyperplane (e.g., a Swiss roll). The disclosure also explores driving a new NDR tailored to manifolds with loops by working over cyclic groups.

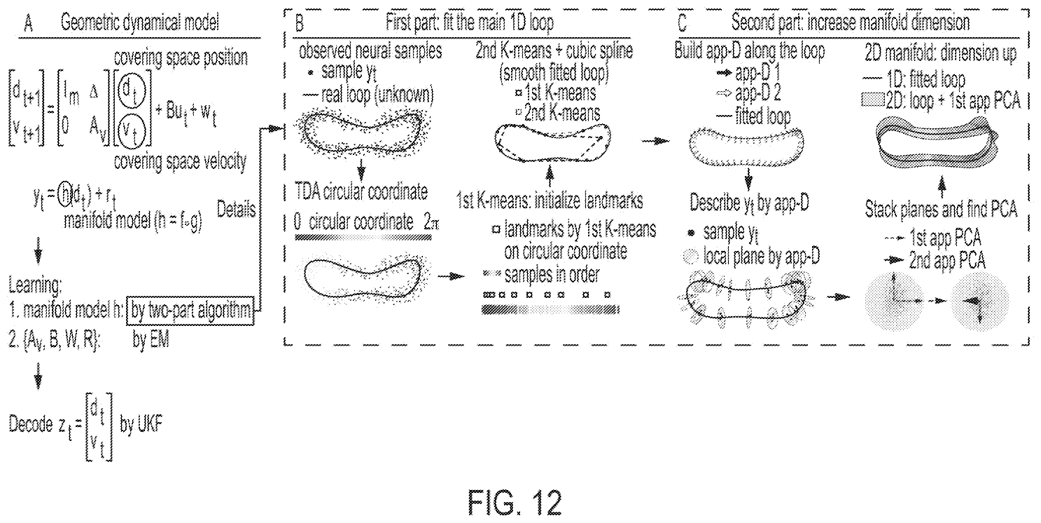

Second, alternatively, ƒ and g may be learned together by defining h=ƒ∘g and learning h directly. For holes, the circular coordinates of each neural data sample may be computed based on its position on 1D loops given by TDA. This can be done on any loopy algebraic structure (e.g., loop, torus). Then, several landmarks may be created by applying K-means clustering on the circular coordinates and then interpolating a curve (or a surface, a volume, etc.) between landmarks with multi-cubic spline or the like. This will provide a smooth analytic function h. If samples y_{1:T} are not close to the manifold, this deviation will be modeled in h by computing a local coordinate system at each point on the manifold—which may be referred to as “appending dimensions”—with the Gram—Schmidt algorithm for finding orthogonal bases. An example based on existing NHP data is provided below and the process is detailed in FIG. 12.

A behavioral observation kₜ of the brain state xₜ may be available, e.g., joint angles during movement or a self-reported mood. Since the nonlinearity is already captured in the dynamic model in Equations 1-3, the disclosure may learn the relationship between the brain state xₜ and behavior as a linear regression in a training dataset as shown below in Equation 7. The disclosure may also learn a nonlinear transform between the state and behavior using for example support vector regression methods or the like.

kₜ=Hxₜ+noise Equation 7:

The third step is to build the geometric decoder and controller. Control of nonlinear neural dynamics has remained elusive to date mainly due to lack of precise neural dynamic models. Enabling control of nonlinear dynamics is a major innovative power of the geometric approach described herein and of the idea of covering space.

What if the dynamic model is not written on the nonlinear manifold? For nonlinear modeling, if PCA is used to go to [symbol] ^{k}, it may be necessary to build a highly nonlinear dynamic model for neural activity over [symbol] ^{k} directly, increasing the difficulty significantly. First, there is no general robust form for modeling a nonlinear system or an associated learning algorithm. Second, designing a controller for a nonlinear system over a manifold is not tractable especially for moderate/high dimensions (related to differential geometry/Lie groups). The geometric approach of the present disclosure provides a major departure from current methods: as the identified manifold captures the nonlinearity already, the dynamic model may be written in a much simpler form over the covering space for this nonlinear manifold, i.e., in terms of zₜ and using the linear model in Equation 1. This simple form makes control tractable despite accounting for complex nonlinearities. The geometric dynamic model of Equations 1-3 will be used to decode or control the brain state xₜ based on neural activity y_{1:t} in real time, but by operating on zₜ on the covering space instead of xₜ on the manifold (as shown in FIG. 7).

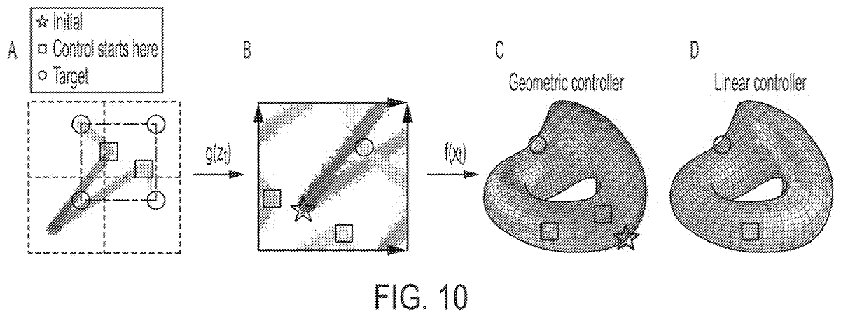

FIG. 9 illustrates an exemplary implementation of using the dynamic model of Equations 1-3 to decode and control the brain state based on neural activity. The implementation shown in FIG. 9 uses a system similar to the system 200 of FIG. 2. In particular, neural activity 602 may be recorded or sampled from a brain 600. A geometric decoder 604 may receive the neural activity 602 and the geometric dynamic model 606 (of Equations 1-3) and may generate a decoded brain state 612. A geometric controller 608 may receive the geometric dynamic model 606, the decoded brain state 612, and a target brain state 610 and may generate control signals 614 for stimulating the brain 600 to achieve the target brain state 610.

To decode, zₜ will be estimated from y_{1:t} by deriving a recursive Bayesian filter consisting of for example either an unscented Kalman filter (UKF) or a particle filter (PF) for Equations 1-3 and 7 to denoise the data as also depicted in FIG. 12 as an example. Also, model-based controllers of network activity (currently lacking) may be derived by taking the decoded brain state as feedback and using the nonlinear dynamic model of Equations 1-3 and 7 (FIG. 9). The dynamic model will predict how the brain state will respond to a given stimulation level at current time, thus guiding the controller 608 to adjust the stimulation. As the nonlinearity is captured in the geometry, standard optimal feedback control, such a linear quadratic regulator (LQR) or linear quadratic Gaussian (LQG) controller, or model predictive control (MPC) can be used by operating on the covering space.

Experiments using the systems and methods of the present disclosure were performed. FIG. 10 illustrates results of a numerical example in Monte Carlo simulations to move the firing rate of 3 neurons to a target in FIG. 10, C for two firing rate trajectories. These trajectories and the target can equivalently be represented on the bounded plane (FIG. 10, B) and the covering space (FIG. 10, A). All circles on the covering space are equivalent as they map to the same target in FIGS. 10, B and C. An optimal feedback-controller was designed that solves the problem on the covering space by using the closest target for each trajectory (FIG. 10, A) in the cost function of a linear quadratic Gaussian controller (LQG). Solving the control problem on the covering space moves the firing rate trajectories in FIG. 10, C to the target firing rate triplet on the distorted torus. A linear dynamic model was also fitted to simulated neural activity driven by a white-noise input on the distorted torus, and the corresponding LQG was designed. Even this best linear controller could not reach the target (FIG. 10, D), showing the importance of the nonlinear geometric controller.

The data below suggest that neural activity can be described on manifolds with loops/holes. However, if in some datasets a well-behaved manifold with its covering space is not found (e.g., torus, 3-sphere), four alternative approaches may be taken: (1) Build the dynamic model over the manifold directly, operate on the bounded plane xₜ instead of covering space zₜ, and use tools from Lie groups to design the decoder/controller. (2) Transform the nonlinear dynamic model of xₜ (no zₜ here) on the low-D manifold to a linear dynamic model in a high-D space by combining TDA with the Koopman operator method just introduced in control theory, which exploits user-selected transform functions of state to get the high-D space. Then control may be performed with this linear model. TDA may be used to select the transfer functions (e.g., if there is a loop, use cos θ and sin θ). (3) Build novel linear but adaptive controllers on a low-D hyperplane, the controllers designed to capture nonlinearities through time-adaptation. (4) Incorporating generic nonlinearities, e.g., with spline bases, to build the dynamic model on low-D hyperplanes and build nonlinear decoders and controllers. Also, to benefit from the paradigm, it is sufficient to find the major (not all) manifold loop(s)/hole(s). The other dimensions can be written in [symbol] ^{m} using appending dimensions (discussed above with reference to learning the geometric dynamic model on the manifold). Neural dynamics can be nonstationary. As the manifold type is a fundamental topological neural property (e.g., rotations in motor cortex are preserved across animals), the inventors hypothesize that the manifold type remains unchanged in time. Non-stationarities may be tracked by adapting the dynamic model on the manifold (Equation 1) with Bayesian methods. TDA is then reperformed, and Eq. (3) is refit to test the hypothesis. Finally, dimensionality reduction to the manifold and dynamic filtering denoise brain network activity.

In addition to Monte Carlo simulation experiment in FIG. 10, the inventors applied the paradigm of the present disclosure on real existing brain data from experiments. The existing data included neural activity recorded from non-human primates (NHP) during performing a movement in a motor experiment. The paradigm can also be applied on human data such as neural data from the corticolimbic system or the like. The paradigm can also be applied for online control experiments on human data for regulation of a brain state such as mood or the like.

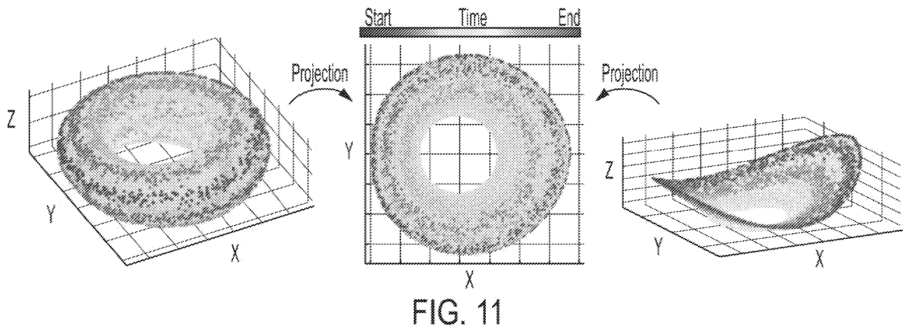

The paradigm was validated on existing data from a motor experiment which was directed to motor cortical spike-field activity during complex naturalistic 3D reach-and-grasp actions. The arm function of this experiment involves the coordinated movement of 27 joints. The inventors ask how neural population activity evolves to produce this movement. While the geometry over which motor cortical activity evolves is unknown, prior studies applied PCA on it during 2D reaches and found that population activity projected on 2D planes evolved with rotations. This may indicate that the geometry involves loops/holes and may present answers regarding the geometry. A major challenge is that many different geometries can result in the same rotations when projected to a 2D plane using PCA, which is shown in FIG. 11. In particular, FIG. 11 illustrates that two 3D trajectories over two different manifold types (a torus and a loopy band) have the same 2D rotational projection. Dot density evolution represents the time evolution. Thus, observing 2D rotations does not specify the geometry in the high-D activity space of tens of neurons; this geometry can be specified by the paradigm of the present disclosure for the first time to elucidate how motor cortical dynamics explain movement.

Most tasks study neural dynamics during 2D or constrained movements. Instead, the inventors studied the above questions using existing neural data recorded during unconstrained 3D movements because they likely cause the neural trajectory to move globally over the manifold rather than get stuck in its local task-specific regions, which will hide the geometry. The inventors applied the paradigm on spike-field motor cortical activity in NHPs performing unconstrained 3D reach-and-grasps to different locations with all 27 joints of the upper-limb measured. The inventors built a geometric model and decoder to estimate joint angles from neural activity. Neural data was existing and previously recorded with a state-of-the-art chronic chamber assembly with 137 penetrating electrodes and an ECoG array in an artificial dura.

First, TDA was applied to identify the manifold type (FIG. 4, A). Second, the model parameters in Equations 1-3 and 7 were learned for the identified manifold (FIG. 4, B, FIG. 12). Third, a nonlinear geometric decoder was built (FIG. 4, C) of the 27 joint angles from spike-field activity. The analysis compares how well the geometric model explains the behavior compared with state-of-the-art linear dynamic models.

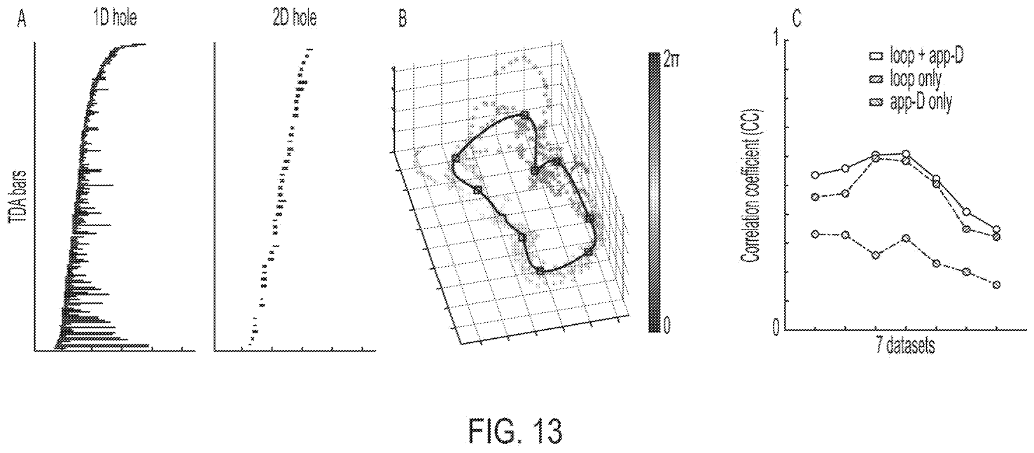

TDA was applied on population spiking activity during reach-and-grasps (shown in FIG. 12 and FIG. 13). TDA finds the Betti number by counting the bottom lines in FIG. 13, A that persist as movement occurs along the horizontal axis. Strikingly, the results illustrated a dominant 1D loop (b₁=1) in all datasets consisting of the firing rates of 137 neurons, despite the high-D neural space and complex multi joint movements. This loop was first fitted using two K-means clusterings and cubic spline interpolation (FIG. 12, B, FIG. 13, B), then its appending dimensions were found (FIG. 12, C) and finally a movement decoder was built based on the position relative to the loop in the local coordinate defined by the loop and the appending dimensions (Equation 7). Surprisingly, this single nonlinear 1D loop in neural activity explained substantially more variance in the movement than all the tens of linear appending dimensions combined (FIG. 13, C). This suggests that the nonlinear loop is a fundamental low-dimensional geometry for motor cortical dynamics and can explain the behavior. For example, as arm movements are feedback-driven, the loop may indicate an integration of neural feedback signals that are input to motor cortex to drive the complex movements.

Backing up, FIG. 12 illustrates how the geometric paradigm works on data. In FIG. 12, A, a dynamic model is learned with expectation maximation (EM) on a loopy manifold h defined also in paragraph [0055] above, which is first learned with a two-part algorithm. FIG. 12, B illustrates that the first part fits the loop. TDA indicates the geometry (loop) and the location of data samples on it by grayscale. The loop is then fit using two K-means clusterings and cubic spline interpolation. FIG. 12, C illustrates that the second part increases the manifold dimension to capture the deviation between the neural samples and the loop. A local coordinate system was derived at each point on the loop by finding a set of orthogonal appending dimensions (app-D). All local coordinates can be stacked to find important directions of deviation using PCA (app PCA) if desired. The model h is now a 2D band as opposed to a 1D loop found by TDA. Appending dimensions were added to increase the manifold dimension.

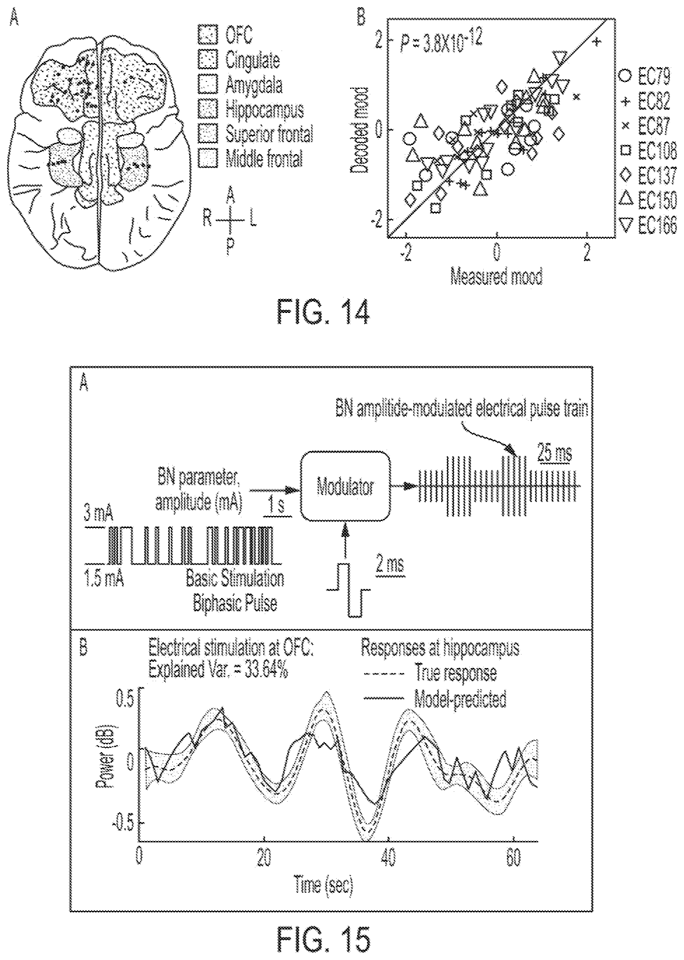

In addition to neural activity underlying movements in the motor experiment, the paradigm can also be used for geometric modeling of human corticolimbic network dynamics underlying mood states or the like. Unlike movements, mood involves multiple functionally-integrated corticolimbic networks including subcortical systems involved in emotion processing (e.g., amygdala) and prefrontal cortical systems involved in implicit and explicit emotion regulation. Given the complexity of network interactions, little is known about multisite network dynamics underlying mood. The geometric paradigm is usable to untangle complex nonlinear dynamics and interactions in human corticolimbic networks and build geometric mood decoders from neural activity. Existing previous multisite corticolimbic ECoG data along with concurrent mood measurements with a validated self-report exists (see FIG. 14). FIG. 14, A illustrates limbic regions that are predictive of mood, and FIG. 14, B illustrates decoding of results from 7 subjects using a neural biomarker, showing feasibility of decoding.

In a breakthrough study, a lab operated by one of the inventors provided the first demonstration that mood variations in humans can be decoded by fitting linear dynamic models of limbic activity after PCA dimensionality reduction (see FIG. 14). However, the modeling had to be restricted to just localized regions in the corticolimbic network as PCA could not sufficiently reduce dimension. So a relatively large part (approximately 50%) of mood variance was unexplained. The inventors hypothesize that the inability of PCA to reduce dimension is because mood-relevant corticolimbic activity moves on nonlinear manifolds (not a hyperplane found by PCA). The geometric decoder can resolve this limitation by capturing a possible nonlinear manifold that may underlie the activity.

The paradigm can also be applied for geometric control of human corticolimbic dynamics underlying brain states such as mood states, pain states, and the like. Precise control of brain network activity with stimulation has not been achieved given the complexity of input-driven neural dynamics. The geometric controller is applicable to this goal by using neural activity as feedback to selectively target specific corticolimbic network ECoG patterns that are neural biomarkers of mood (FIGS. 9 and 14). Stimulation in lateral orbitofrontal cortex (OFC) was recently shown to acutely improve mood in patients with depression symptoms. Also, OFC has connections with several areas implicated in emotion regulation and processing, e.g., amygdala and cingulate cortex, making it well-positioned to modulate their activity. As such, the geometric paradigm can be used to control stimulation in OFC for example, or in any other appropriate brain region.

Input-driven geometric model: Even if spontaneous neural dynamics are modeled, this is not sufficient for control. Control requires prediction of how stimulation input drives activity on the nonlinear manifold, which is relatively challenging. No data-driven model has achieved this prediction. To solve this challenge, the geometric paradigm can be used in experiments in which the brain is stimulated in an appropriate brain region such as OFC. The stimulation can be done as open-loop stochastic multilevel noise (MN) stimulation waveform designed by the lab of an inventor that consists of a pulse train whose amplitude and frequency randomly switch between multiple levels, and thus is theoretically proven to excite network activity (FIG. 15, A). The neural response data to MN can be used to fit the model in Equations 1-3 with input. The input will be the known stimulation parameters here and can be given to the machine learning processor 104 through the input device 108 in FIG. 1. The target network state for the controller can be set as the one that achieves a desired (neutral) therapeutic level of decoded mood, which is referred to as neural biomarker. FIG. 15, A illustrates the MN stimulation when there are two levels referred to as the binary noise (BN) stimulation to learn a linear dynamic model. FIG. 15, B illustrates existing ECoG data previously collected in the human corticolimbic system where OFC is being stimulated with the BN stimulation. FIG. 15, B illustrates that a linear dynamic model can be fitted to this data and the prediction of ECoG band power response to BN with the fitted linear dynamic models. Compared to this previous linear model, the geometric model can additionally incorporate nonlinearity in modeling the effect of stimulation and thus can improve its accuracy.

The inventors previously delivered a two-level binary noise (BN) (FIG. 14, A) to OFC and fitted a linear dynamic model (FIG. 15, B). This explained approximately 35% of the power feature variance. While prediction of neural response with the linear model was possible, there was a relatively large unexplained variance that the inventors hypothesize is due to nonlinearity and the geometric method can mitigate.

Closed-loop control: The geometric paradigm can be used for closed-loop control of a brain state such as mood or pain or the like. The geometric decoder can obtain the decoded mood state, i.e., neural biomarker, and the geometric controller can then use it as feedback to control the stimulation amplitude and frequency to drive it to target (FIGS. 9 and 4, C). The controlled state can be compared with the target and the difference or error used to update the stimulation parameters continuously and optimally using the geometric controller. This closed-loop geometric control can thus allow for precise adjustment and personalization of stimulation compared with open-loop stimulation and on-off closed-loop control that turns stimulation on when the decoded mood (neural biomarker) goes below a threshold.

The inventors have built a closed-loop clinical simulation testbed for stimulation so the hardware/software pipeline already exists in place to test the geometric controller in simulations and in the clinic. To mitigate stimulation artifacts, neural data can be recorded at sample rates greater than 10 kilohertz (kHz) to enable template-based artifact removal, or controller can alternate between sense and stimulation in short intervals.

The present disclosure provides many areas of innovativeness, a few of which are described below.

First, enabling control of nonlinear brain network dynamics using the fundamental concepts of geometry and covering space provides a major paradigm shift for neural modulation research with broad application in basic and clinical neuroscience. No control strategy is currently available with any comparable level of precision.

Second, despite huge potential, closed-loop stimulation technology for neuropsychiatric disorders has not been developed as these disorders involve a distributed corticolimbic network whose selective modulation will require innovative methods to model and control the barrage of complex neural activity as achieved by the geometric paradigm.

Third, while TDA has been used for qualitative data description and for classification/decoding, no method exists to link TDA to dynamic modeling or control. This requires going beyond TDA, which finds the number of major loops and holes, and developing new methods as achieved by the geometric paradigm to also (a) increase the manifold dimension by learning a model for the manifold (FIG. 12, B) and building local coordinates on it (FIG. 12, C), (b) learn an analytical dynamic model on the manifold (FIG. 12, A), (c) enable control by combining the idea of covering space in topology with stochastic control for the first time (FIG. 4). The new methods will contribute not only to neuroscience but also to data science and, given the complexity across biology, to other biological modeling and control problems.

Fourth, even for neural dynamic modeling alone, the geometric paradigm solves the challenge of building a model that is accurate yet interpretable, low-dimensional, and controllable so that it can be used to interpret the neural basis of behavior and design neurotechnology. In contrast, current methods either build complex neural networks that are hard to interpret and not controllable, or linear models that cannot capture nonlinearity.

Fifth, this paradigm will lead to fundamental discoveries about the neural control of movement, which is incompletely understood. Unlike prior work that project high-D activity on low-D planes, the inventors find the geometry in high-D motor cortical activity space for the first time. Also, unlike most work that study constrained movements, the inventors have demonstrated the paradigm on neural data during unconstrained 3D movements to reveal the global structure of neural dynamics.

Where used throughout the specification and the claims, “at least one of A or B” includes “A” only, “B” only, or “A and B.” Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

1. A method for nonlinear modeling, decoding, and control of neural dynamics, the method comprising:
identifying, based on neural time-series samples, a type of a manifold as a base for a neural model;
learning, based on a covering space, a dynamic model that is fit on the manifold to create the neural model; and
creating, using the neural model, a geometric decoder and a geometric controller.

2. The method of claim 1 wherein identifying the type of manifold includes counting a quantity of persistent holes or loops in the neural time-series samples using topological data analysis (TDA).

3. The method of claim 2 wherein counting the quantity of the persistent holes or loops using the TDA includes computing Betti numbers.

4. The method of claim 1 wherein learning the dynamic model includes learning a covering map, learning a function indicating how the manifold is embedded in a space of neural activity, and finding parameters of the dynamic model on the covering space.

5. The method of claim 4 wherein learning the covering map includes learning the covering map based on the type of manifold.

6. The method of claim 4 wherein finding the parameters of the dynamic model includes a new unsupervised expectation-maximization (EM) method.

7. The method of claim 4 wherein learning the function includes learning a first portion of the function that maps undistorted coordinates to neural data, and a second portion of the function that maps a manifold state of the type of manifold to an embedding space.

8. The method of claim 7 wherein learning the second portion of the function includes learning the second portion based on the type of manifold.

9. The method of claim 7 wherein learning the first portion of the function includes using nonlinear dimensionality reduction (NDR) and combining the NDR with support vector methods of functional approximation of various kernels.

10. The method of claim 4 wherein learning the function includes learning a composition of the function and the covering map.

11. The method of claim 10 wherein learning the composition of the function and the covering map includes computing circular coordinates of each neural data sample based on its position on a one-dimensional loop given by topological data analysis (TDA).

12. The method of claim 11 wherein learning the composition of the function and the covering map includes computing several landmarks by applying K-means clustering on the circular coordinates of the neural data samples and interpolating a curve between two or more of the several landmarks with a spline.

13. The method of claim 12 wherein a local coordinate system is computed at each point on a loop with a Gram—Schmidt algorithm where the local coordinate system provides appending dimensions to increase a manifold dimension of the manifold.

14. The method of claim 13 wherein directions of variations of the neural data samples on the manifold are found using a dimensionality reduction method.

15. The method of claim 14 wherein the dimensionality reduction method includes principal components analysis (PCA).

16. The method of claim 1 wherein creating the geometric decoder and the geometric controller includes creating the geometric decoder to decode a brain state based on neural activity in real time.

17. The method of claim 16 wherein creating the geometric decoder to decode the brain state further includes estimating a D-dimensional state on the covering space from neural data.

18. The method of claim 17 wherein the geometric decoder includes a Bayesian filter constructed for the dynamic model including at least one of a particle filter or an unscented Kalman filter.

19. The method of claim 17 wherein the geometric decoder is configured for regressing neural data samples to the manifold by finding a closest point to each sample on the manifold.

20. The method of claim 17 wherein behavior is decoded as a linear or nonlinear function of a decoded state on the covering space.

21. The method of claim 16 wherein creating the geometric decoder and the geometric controller includes creating the geometric controller by taking the decoded brain state as feedback and using the dynamic model.

22. The method of claim 21 wherein the dynamic model is configured to predict a change in the brain state in response to a given stimulation input level at a current time.

23. The method of claim 21 wherein the geometric controller includes at least one of an optimal linear quadratic regulator, a linear quadratic Gaussian controller, or a model-predictive controller on the covering space built using the dynamic model.

24. A system for nonlinear modeling, decoding, and control of neural dynamics, the system comprising:
at least one of an input device or a sensor configured to receive neural time-series samples; and
a processor coupled to the at least one of the input device or the sensor and configured to:
receive or determine a type of manifold to use as a base for a neural model,
learn a dynamic model that is fit on the manifold to create the neural model based on a covering space, and
create a geometric decoder and a geometric controller using the neural model.
